(19) United States
(12) Patent Application Publication
(10) Pub. No.: US 2005/0266499 A1
(43) Pub. Date: Dec. 1, 2005

(54) Method and apparatus for testing for presence or absence of select biological substances

(75) Inventors: Sze Wee Tan, Singapore (SG); John Rodney Warmington, Leeming (AU); Aaron John Mackay, Thornlie (AU); Ai-Teck Tan, Singapore (SG)

Correspondence Address: KUDIRKA & JOBSE, LLP, ONE STATE STREET, SUITE 800, BOSTON, MA, 02109, US

(73) Assignee: Rockeby biomed Corporation, LTD., Singapore (SG)

(21) Appl. No.: 11/186,289

(22) Filed: Jul. 21, 2005

Related U.S. Application Data
(63) Continuation-in-part of application No. 10/682,807, filed on Oct. 10, 2003.
(63) Continuation-in-part of application No. 09/841,188, filed on Apr. 25, 2001, now Pat. No. 6,916,626.
(60) Provisional application No. 60/665,824, filed on Mar. 28, 2005.
(60) Provisional application No. 60/676,248, filed on Apr. 29, 2005.

(51) Int. Cl. G01N 33/53; C12M 1/34

(52) U.S. Cl. 435/7.1; 435/287.2

(57) ABSTRACT

A fluid flow test device comprising: a chromatography path of aqueous wicking material on which is deposited at an upstream site at a selected delivery time an aqueous biological sample to be tested for the presence or absence of a target material; a capture material bound to the wicking material at a test site downstream of the upstream site, the capture material having a functionality that binds to a first locus on the target material upon wicking of the target material downstream to the test site; a visual label material having a functionality that binds to a second locus on the target material, the chromatography path having a site upstream of the test site at which the visual label material is begun to wick at a predetermined point in time after the first delivery time such that the visual label material does not intermix with the aqueous biological sample within the chromatography path.

### RELATED APPLICATIONS

This application claims the benefit of priority under 35 U.S.C. Section 119 to U.S. provisional application Ser. No. 60/665,824 filed Mar. 28, 2005 and 60/676,248 filed Apr. 29, 2005 the disclosures of both of which are incorporated herein by reference in their entirety as if fully set forth herein. This application is a continuation-in-part of and claims the benefit of priority under 35 U.S.C. Section 120 to U.S. application Ser. No. 10/682,807 filed Oct. 10, 2003 and Ser. No. 09/841,188 filed Apr. 25, 2001 the disclosures of both of which are incorporated herein by reference in their entirety as if fully set forth herein.

### FIELD OF THE INVENTION

The present invention relates to methods and apparati for testing a sample of biological fluid for the presence or absence of a selected substance and more particularly to testing for the presence or absence of antibodies and their corresponding antigens.

### BACKGROUND OF THE INVENTION

A variety of testing devices are known that use what has been termed “lateral flow” technology to separate cell, solid materials, certain large molecules and the like from selected substances of interest in a biological sample. Such prior devices typically comprise a single linear or planar, thin elongated generally horizontally disposed strip or column of chromatography material that wicks aqueous materials along the length of the continuous strip of wicking/chromatography material. An aqueous sample of biological fluid to be tested for the presence or absence of a selected antigen material is applied to a discrete spot on the horizontally disposed column/strip of chromatography material. The applied sample is then wicked laterally along the length of the column in a lateral direction toward a sink. A test site is located between the sample application site and the sink such that when a sample of aqueous fluid is applied to the sample application site, the sample must first pass by and make contact with the test site. A known antibody to a selected antigen of interest is bound to the matrix of the chromatography material at the test site so that any antigen to the antibody in the sample is captured and bound at the test site. A visually identifiable material that selectively binds to the antigen of interest in the sample is applied to the chromatography strip at the same time as the biological fluid is applied to the same site as the sample is applied. The visual label is alternatively pre-deposited on the strip at the sample application site or at a site slightly downstream of the sample application site/position. Additional drops of aqueous buffer are applied to the sample application site to cause the sample material and the visual label material to wick simultaneously together in the same body of fluid toward the test site. The visual label material typically binds specifically to the antigen of interest that is present in the sample upon intermixing of the visual label material and the sample. The bound antigen/label are wicked downstream together to the test site. The visual label material is reaction-specific to the one preselected sample antigen of interest and thus other chemical entities that may be present in the biological sample do not bind to or deplete the visual label material.

### SUMMARY OF THE INVENTION

The present invention comprises an apparatus and method for testing for the presence or absence of a selected biological substance. The selected biological substance can be, for example, a preselected antibody, antigen, protein, enzyme or any molecule, cell or biological moiety (hereinafter individually and collectively “analyte”) that can be wicked along and through a series of separate, but fluidly communicating, wicking components that are placed in overlapping wicking engagement with each other. The analyte is captured on the chromatography medium by a capture substance that is bound to the chromatography medium at a test site. The test site is located at a discrete predetermined location along the path of wicking of the analyte. In one embodiment, a complementary antigen that is reactive with the select antibody is used as the capture material at the test site. And, vice versa, a select antigen can be tested for by using a complementary antibody that is reactive with the select antigen as the capture substance at the test site.

The invention further provides a two step chromatography, elution or aqueous chase process. First, the biological sample is delivered to a sample application site on a chromatographic pathway and wicked/chased toward the position of a capture test site. Next, in a second step subsequent to the time that wicking of the sample material has been started/initiated, a visual label material is wicked along the chromatography path toward the test site such that the visual label material does not contact or engage with the sample material at any time before reaching the location of the capture test site. The visual label material either reacts with the sample in its unbound configurational form or in the configurational form that the sample material assumes after it reacts with the capture material at the test site. The sample material is typically wicked all the way to the position of the test site before the visual label material is applied to the chromatography path.

In another aspect of the invention, a test device is provided that has two chromatographic or wicking pathways, one pathway for wicking of the sample material to the location of the test site and another pathway being at least partially physically separate from the first pathway for wicking the visual label material to the test site.

In accordance with the invention there is provided a fluid flow test device comprising:
a first strip of aqueous wicking material on which is deposited at a first delivery time an aqueous biological sample to be tested for the presence or absence of a target material;
a second strip of aqueous wicking material having a test site that contains a capture material that binds to the target material, the second strip being in fluid communication with the first strip of aqueous wicking material at a first location upstream of the test site;
a visual label material that is wicked along the second strip of material from a location upstream of the test site to the test site beginning at a predetermined point in time after the first delivery time.

The device can include a third strip of aqueous wicking material that is in fluid flow communication with the second strip at a second location upstream of the test site, the visual label material being delivered to the second strip from the third strip for aqueous wicking delivery to the second strip at the predetermined point in time after the first delivery time. The target material is typically a selected antibody and the capture material a selected antigen that complexes the selected antibody. The visual label material is either applied to the third strip of material at the time of use of the test device or the visual label material is pre-applied to third strip of material and embedded therein before use of the test device.

Further in accordance with the invention there is provided a method of testing for the presence or absence of a target material in an aqueous biological sample, comprising:
delivering the aqueous biological sample to a first strip of aqueous wicking material at a first selected delivery time;
providing a second strip of aqueous wicking material having a selected capture material stationarily located at a test site, the capture material being capable of binding to the target material, the first strip of material being in fluid communication with the second strip at a first selected location on the second strip that is upstream of the test site;
delivering a visual label material to the second strip of material upstream of the test site, the visual label material being delivered to the second strip of material at a second predetermined point in time after the first selected delivery time.

The second predetermined point in time is preferably long enough after the first selected delivery time to ensure that the biological sample has been wicked downstream to the test site.

The target material is a selected antibody that binds to the visual label material, the visual label material comprising an antigen that non-selectively binds to the selected antibody and to other antibodies that are contained in the biological sample. The visual label material can alternatively comprise an antibody to the target material that binds to the target material. The capture material typically comprises an antigen that selectively binds to the antibody.

Preferably, the visual label material is applied to a third strip of aqueous wicking material that is in fluid communication with the second strip at a second location that is upstream of the first location. A separation distance is preferably selected between the first and second locations such that the biological sample is ensured of being wicked downstream to the test site before the visual label material is wicked downstream to the test site. The visual label material is typically delivered to a second location on the second strip that is upstream of the first location.

In another aspect of the invention there is provided, a method of testing for the presence or absence of a target material in an aqueous biological sample, comprising:
binding a selected capture material to a test site on a strip of aqueous wicking material at a location downstream of a selected application site on the strip, the capture material being capable of binding to the selected target material;
delivering the aqueous biological sample to the selected application site at a first selected delivery time, and allowing the biological sample to be wicked downstream toward the test site;
delivering a visual label material to the strip of material at a location upstream of the test site for downstream wicking of the visual label material to the test site, the visual label material being delivered to the strip of aqueous wicking material at a second predetermined point in time that occurs after the first selected delivery time.

Further in accordance with the invention there is provided, a fluid flow test device comprising:
a first strip of aqueous wicking material on which is deposited at a first delivery time an aqueous biological sample to be tested for the presence or absence of a target material;
a second strip of aqueous wicking material having a test site that contains a capture material that binds to the target material, the second strip being in fluid communication with the first strip of aqueous wicking material at a first location upstream of the test site;
a third strip of aqueous wicking material in fluid flow communication with the second strip at a second location upstream of the test site, a visual label material being deposited on the third strip for wicking along the second strip of material to the test site.

The visual label material can either be applied to the third strip of material at the time of use of the test device or the visual label material is pre-applied to third strip of material and embedded therein before use of the test device.

Further in accordance with the invention there is provided a fluid flow test device comprising:
a chromatography path of aqueous wicking material on which is deposited at an upstream site at a selected delivery time an aqueous biological sample to be tested for the presence or absence of a target material;
a capture material bound to the wicking material at a test site downstream of the upstream site, the capture material having a functionality that binds to a first locus on the target material upon wicking of the target material downstream to the test site;
a visual label material having a functionality that binds to a second locus on the target material, the chromatography path having a site upstream of the test site at which the visual label material is begun to wick at a predetermined point in time after the first delivery time such that the visual label material does not intermix with the aqueous biological sample within the chromatography path. The chromatography path typically comprises a first path extending between the test site and the upstream site at which the sample is applied and a second path at least partially physically separate from the first path extending between the test site and the upstream site at which the visual label material is applied.

### BRIEF DESCRIPTION OF THE DRAWINGS

The above and further advantages of the invention may be better understood by referring to the following description in conjunction with the accompanying drawings in which:

FIG. 1 is a top perspective view of a fully assembled test apparatus according to the invention;

FIG. 2 is an exploded view of the FIG. 1 apparatus

FIG. 3 is a side cross-sectional view along lines 3-3 of FIG. 1;

FIG. 4 is a sectional view along lines 4-4 of FIG. 3;

FIG. 5 is a side view of a chromatographic subassembly of the FIG. 1 apparatus shown in an assembled working array; and,

FIG. 6 is a side exploded view of the chromatographic subassembly of FIG. 5 showing one specific example of an exact relative horizontal and vertical arrangement of components of the chromatography subassembly.

### DETAILED DESCRIPTION

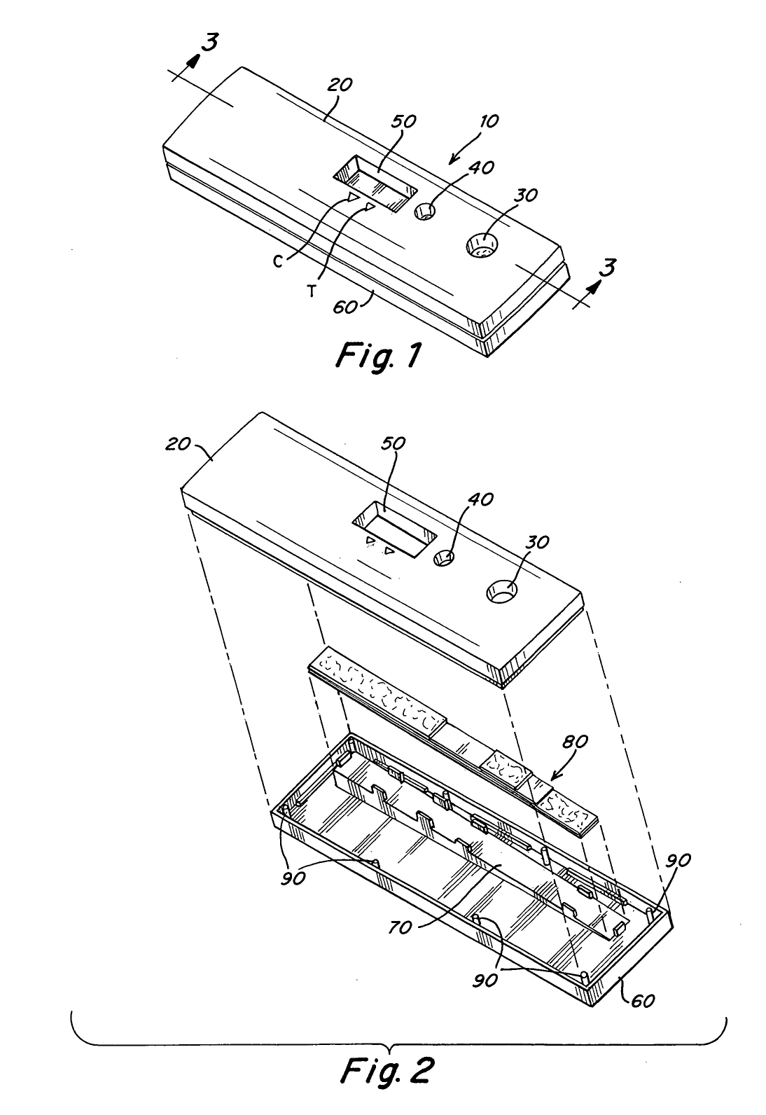

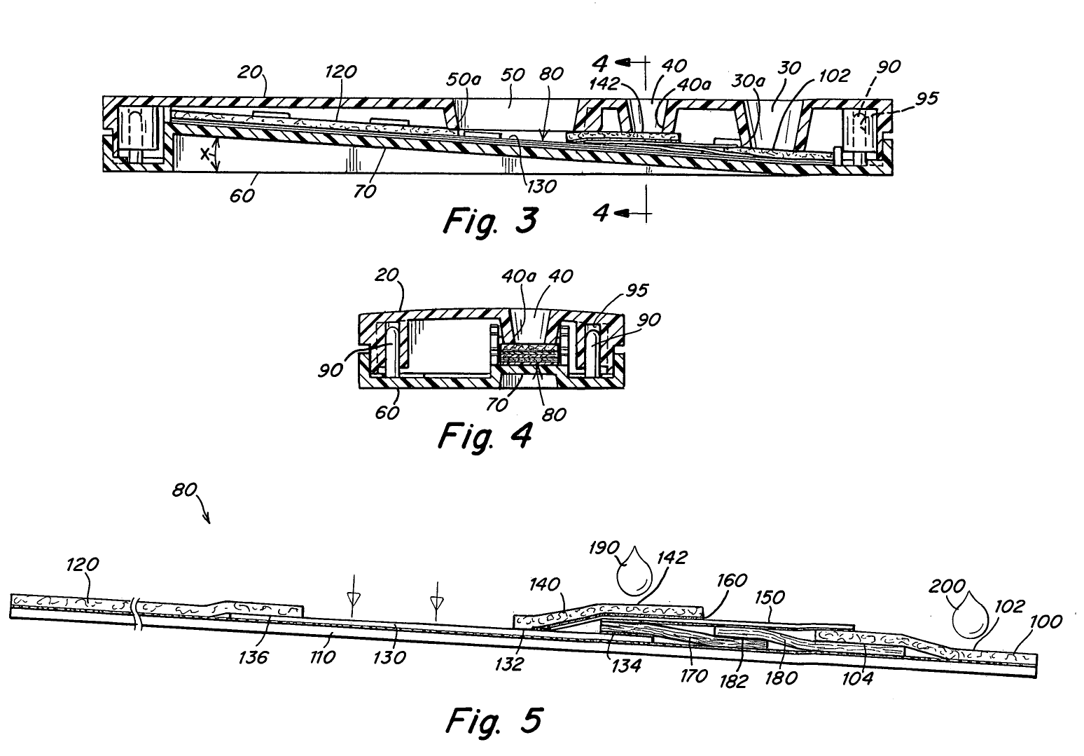

FIG. 1 shows a fully assembled embodiment of a test apparatus 10 according to the invention. The apparatus 10 comprises a top housing component 20 and bottom housing component 60 that readily snap or snug fit together via pins 90 and complementary apertures 95, FIGS. 3, 4 to hold the top 10 and bottom housing components firmly together. As can be readily imagined the pins 90 are also readily disengageable from apertures 95 to enable the entire apparatus to be readily disassembled as shown in FIG. 2. As shown, a chromatographic subassembly 80 is mounted between the housing components 10, 20 on a slightly inclined ramp 70 that is formed as an integral portion of the bottom housing component 60. The ramp 70 inclines upwardly at an angle X to horizontal, FIG. 3, from the upstream end, the sample application end, toward the other downstream end of the chromatographic subassembly 80. The plastic housing components 10, 20, and the integral ramp 70, comprise a resilient but rigid plastic material that provides structural stability to the apparatus 10 and which can be injection or compression molded, is inert and impervious to water and the buffer 200 and sample 190 materials generally. As shown in the cross section in FIG. 4, the assembly 80 is sandwiched and firmly held on the surface of ramp 70 by the bottom surfaces of sidewalls 30a, 40a, 50a that surround apertures 30, 40, 50 respectively, the depth of the sidewalls being selected to mate under slight compression with the corresponding underlying portion of the upper surface of assembly 80 when top housing 20 is snap fit together with bottom housing component 60.

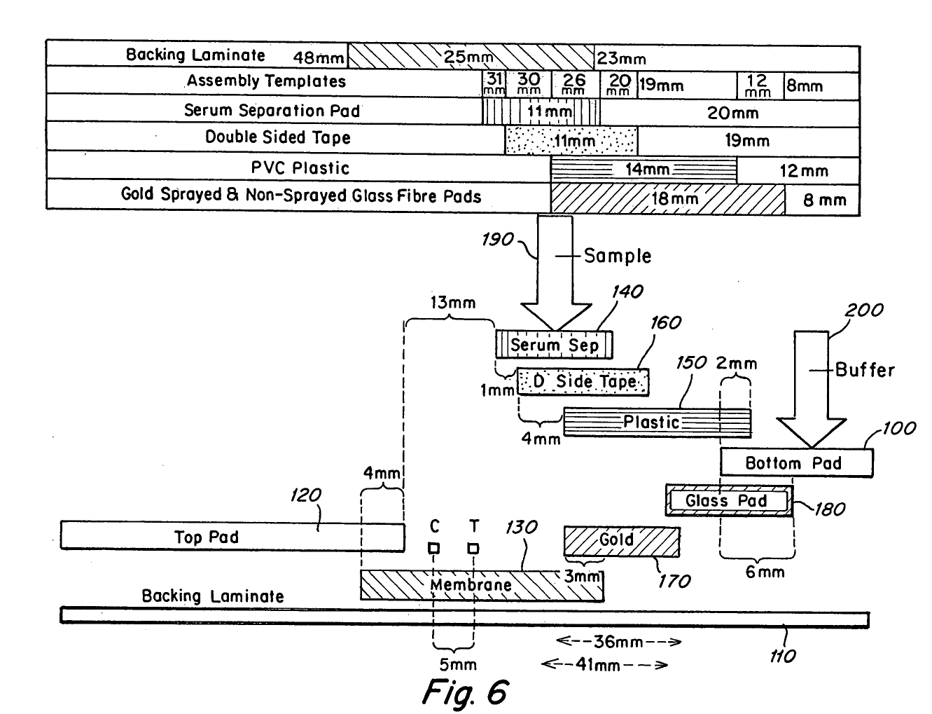

As best shown in FIGS. 5, 6 the chromatographic strip, column or assembly 80 is comprised of an elongated base strip of bendable, but stiff, inert plastic 110 that supports on its top side a multiplicity of other components (held in place by adhesive on the top surface of the strip 110) namely, (a) a chase fluid (typically aqueous buffer fluid) 200 application wicking pad 100 located at the most upstream point/location of the strip 110, (b) a glass fiber wicking pad 180 (immediately downstream of pad 100) that is in fluid communication with pad 100 at mating surfaces 104, (c) a second glass fiber wicking pad 170 (immediately downstream of pad 180) containing within its matrix a visual label-analyte binding material (e.g. a protein coupled to/conjugated with gold, the gold providing a visually identifiable color when accumulated at the test and control sites T, C), pad 170 being in fluid communication with pad 180 at mating surfaces 182, (d) a wicking membrane or pad 130 (immediately downstream of pad 170) being in fluid communication with pad 170 at mating surfaces 134 and also in downstream fluid communication with pad 140 at mating surfaces 132, (e) a sample or specimen 190 application pad 140 that is insulated from being in direct fluid communication with any of pads 100 or 180 or 170 by an intervening strip of inert water impervious plastic 150, the pad 140 being in fluid communication with wicking membrane 130 at mating surfaces 132 which are downstream of mating surfaces 134, (f) a wicking top pad located furthest downstream in fluid communication with membrane 130 at mating surfaces 136. The wicking membrane is typically comprised of a nitrocellulose membrane material.

A strip of water impervious double sided tape 160 is disposed between the sample application pad 140 and the plastic barrier 150 to ensure that the pad 140 is firmly held in position relative to the other underlying components of the assembly 80 as shown in FIGS. 5, 6. As can be readily imagined the precise spacing and distances shown in FIG. 6 can be varied and arranged in alternative as needed or desired to achieve the two step sample application and visual label elution process of the invention. As shown an aqueous sample/specimen 190 is applied to a sample application spot 142 on the pad 140. The aqueous sample 190 comprises biological fluid such as blood, urine, perspiration, tear or other aqueous biological fluid that would normally contain a predetermined biological molecule, complex of molecules or other molecular sized moiety/material (“analyte”) that indicates the existence of a selected condition in the subject. The presence or absence of the predetermined analyte in the subject from whom the fluid is taken can be tested for by binding the analyte to a material that captures the analyte at a test site T. The capture material is bound to the medium of the membrane 130 at the test site T.

As shown in a specific embodiment in FIG. 6 for purposes of explanation, the various pads are laterally and vertically positioned/aligned relative to each other by the distances illustrated for purposes of achieving the separation, filtering and timing of fluid travel as described. As can be readily imagined that alignment, lengths and arrangement may be varied to suit the preferences of the user and the specific analyte to be tested for.

The sample application spot 142 is aligned directly under a sample application aperture 40 provided in the housing component 20. In practice, the sample 190 is first applied to spot 142 and allowed for a relatively brief amount of time, e.g. 1-10 seconds, to wick in a downstream direction, i.e. wicking occurs from right to left as shown in FIGS. 5, 6. Once applied to spot 142, the aqueous sample 190 wicks through the mating point/surfaces 132 of pad 140 and continues to wick in a downstream direction through membrane 130 to and through test site T and control site C on membrane 130. Housing component 20 is provided with an aperture 50 with which test site T and control site C are positionally aligned for viewing through aperture 50 when the assembly 80 is positioned on ramp 70 and the apparatus 10 is fully assembled as shown in FIG. 1.

A predetermined amount of time after the sample 190 has been applied to spot 142, and preferably after the sample 190 has been allowed to wick to the location of test site T, an aqueous buffer 200 that does not contain analyte material is applied to spot 102 on upstream pad 100. Spot 102 is positionally aligned with an aperture 30 provided in housing component 20 for ready and precise application of the buffer 200 to the pad 100. When applied, the buffer 200 wicks continuously downstream first through pad 100, then through mating surfaces 104 to and through glass pad 180 then through mating surfaces 182 to and through visual label containing pad 170, then through mating surfaces 134 to and through membrane 130 past the position of both test site T and control site C and then through mating surfaces 136 to sink pad 120. Pad 120 is relatively larger in volume than the other pads such that it attracts aqueous fluids 190 and 200 that are applied at the upstream end of the stepped chromatography paths leading to pad 120, the pad 120 thus acting as a sink to cause fluids 190 and 200 to tend to travel in the downstream direction, i.e. right to left as shown in FIGS. 3, 5, 6.

Typically a substantially greater volume of buffer 200 is applied to spot 102 than the volume of sample material 190 that is applied to spot 142, e.g. 2-10 times the volume. Pad 170 contains a selected amount of a predetermined visual label material that is capable of binding to the selected analyte material, i.e. capable of binding either directly to the analyte in its free unbound form or capable of binding to the analyte in its bound form after being captured by the capture material resident at the test site T. A large volume of buffer 200 is applied to spot 102 (relative to the volume of sample 190) so that there is a sufficient, if not excess, amount of buffer available to assure that the visual label material is fully dissolved or otherwise entrained in the buffer and wicked completely downstream along the chromatography path from pad 170 to test site T and control site C.

In the sequence of steps described, the sample 190 is first wicked downstream through pad 130. Only after the sample 190 has been fully wicked to at least the downstream location of test site T is the buffer 200 and the visual label material resident in pad 170 then wicked along at least the same chromatography path through pad 130 to the location of test sites T and C. The precise amount of time in sequencing the application of sample 190 and buffer 200 and the precise volume of sample 190 and buffer 200 that is applied can be varied depending on the precise length and number of separate pads that comprise the chromatography paths between the sample application spot 142 and the test site T and between the location of the visual label pad 170 and the test site T. In any case, the sample material 190 is wicked at/during such a period of time and along such a path that the visual label material does not mix or combine with the sample at any time within the matrix of the chromatography path prior the time that the sample 190 has wicked to the location of test site T.

The materials of which the pads 100, 120, 140, 170, 180, and membrane 130 are comprised are capable of readily wicking aqueous fluid. The mating surfaces 104, 182, 134, 132, 136 are arranged such that opposing surfaces of the respectively mating pads are engaged sufficiently with each other to enable aqueous fluid to readily wick from one pad to another with which it is mated. The overlapping of separate generally planar, but bendable pads, 100, 180, 170, 140, 130, 120 creates a wicking or chromatography path between the pads that is both continuous and interrupted or stepped from horizontal to vertical at mating surfaces 104, 182, 134, 132 and 136. Extraneous materials contained within the buffer 200 or the sample 190 that can interfere with the reaction between the analyte and the visual label material or the capture material at the test site T can be selectively filtered by both the matrices of the chromatography pads themselves and by the mating surfaces that make up an interrupted or stepped chromatography path. The sample application pad 140 initially filters cells out of the aqueous flow that wicks through to the test site T. The interrupted or stepped chromatography path as specifically shown and described herein can be varied in many ways, e.g. by varying the number and length of mating surfaces and chromatography pads such that the overall configuration and length of the chromatography path and the number of mating surfaces is tailored to achieve any desired degree of filtering or separation of sample from the visual label. As shown best in FIGS. 5, 6, the downstream chromatography flow path of the sample 190 is physically separated from the downstream chromatography flow path of the visual label material by the plastic strip 150 and by the arrangement of pads and mating surfaces, the pads providing a generally horizontal path of flow and the mating surfaces providing a generally vertical path of flow, the combination of horizontal and vertical flow forming a stepped or interrupted chromatography path of flow. Although pad 140 is in fluid communication with pad 170 via mating surface 132, membrane 130 and mating surface 134, flow through pads 130 and 140 tends to travel downstream instead of upstream by virtue of pad 120 acting as a sink that attracts fluid flow.

The visual label material resident in pad 170 and the capture material resident at test site T are typically preselected to be reactive with and bind to the predetermined analyte being tested for. Alternatively, the visual label material may be selected to be reactive with the reaction product of the analyte and the capture material. Thus when the visual label material is wicked to the location of the test site T, the visual label material will be captured and accumulate at the test site (being visible to the eye indicating the presence of analyte in the sample) only when the analyte has been captured at the test site T. To ensure that the visual label material is in fact responsible for a positive visual presence at the test site T, a control capture substance that reacts with and captures the visual label material is bound to the membrane 130 at a control site C located downstream of the test site T. The visual label material is provided in a molar amount on pad 170 in excess of the maximum molar amount of capture material present at test site T such that upon wicking of the visual label material to the point of the test site T an excess of amount of visual label material is guaranteed to flow past test site T to the location of the control site C. If, after application of sufficient buffer 200 to cause the visual label material to wick to the point of control site C, a positive visual identification of the visual label material can be made at control site C, then the user of the device is assured that a positive identification of visual label material present at the test site is not a false positive from an extraneous substance binding to the test site T and that the device is working correctly.

In one specific embodiment of a test device according to the invention, the sample analyte comprises an antibody to a selected condition or antigen present in the biological subject, e.g. an immunoglobulin (IgG, IgM, IgA, IgE, IgD) and variants thereof; and the capture material comprises one or more antigens to the antibody. And, the visual label material comprises an antibody or antigen to the selected analyte-antibody

For example the analyte could comprise a specific anti-condition X IgG, such as anti-Candida immunoglobulin G (“IgG”). In such an example, the capture material that is bound to the matrix of the membrane at test site T comprises one or more antigens (typically proteins) to the anti-Candida IgG. When the anti-Candida IgG containing sample 190 is wicked from the sample application site 142 to the test site T, at least one antigen at the test site has an epitope which is recognized by the antibody and to which the antibody binds. Once the anti-Candida antibody is bound to the antigen at the test site T, the antibody unfolds exposing another site on the antibody that is reactive with a visual label protein that recognizes the exposed reactive site on the bound antibody. In such a case, the visual label material can be selected to comprise a gold conjugate of another different protein reactive with the anti-Candida IgG, the visual label material typically also being reactive with any IgG that may be present in the sample and not just with anti-Candida IgG. In such an example, the visual label material must be separated from contact with the sample 190 within the chromatography path prior to the sample's being wicked to the test site T in order to avoid the visual label material being depleted by IgG other than the specific anti-Candida IgG (or other specific anti-X IgG) whose presence or absence is being tested for. Separation of the generally reactive visual label material from the sample 190 is achieved by sequenced timing of application of the sample 190 and the buffer 200 as described above and by physical separation of the application pad 140 from the visual label impregnated pad 170 as described above.

In the specific embodiment described regarding anti-Candida IgG, the capture material resident at the test site T can comprise a mixture of one or more antigens derived from a cell line of one or more specific Candida species (e.g. C. albicans, C. tropicalis, C. parapsilosis, C. Iusitaneae, C. glabrata and C. krusei) and is reactive with antibodies to one or more or all of such species. The antigens are sprayed on and bound to the membrane at the test site T. As can be readily imagined the test and control line capture materials can comprise antigens derived from and peculiar to any preselected biologic subject's condition and the substance/analyte to be tested for in the sample 190 can be the antibody to the antigen that is peculiar to any such preselected condition. A visual label material that is either reaction specific to the analyte or generally reactive with the class of molecules of the analyte can be used.

Alternatively, the material selected for binding to the test site T can comprise one or more antibodies to a preselected biologic subject's condition (instead of antigen) and the analyte can comprise one or more antigens (instead of antibody) peculiar to the condition. In such an embodiment, the visual label material is selected to be reactive with the antigen, e.g. another different antibody to the antigen.

1. A fluid flow test device comprising:
a first strip of aqueous wicking material on which is deposited at a first delivery time an aqueous biological sample to be tested for the presence or absence of a target material;
a second strip of aqueous wicking material having a test site that contains a capture material that binds to the target material, the second strip being in fluid communication with the first strip of aqueous wicking material at a first location upstream of the test site;
a visual label material that is wicked along the second strip of material from a location upstream of the test site to the test site beginning at a predetermined point in time after the first delivery time.

2. The fluid flow test device of claim 1 wherein the device includes a third strip of aqueous wicking material that is in fluid flow communication with the second strip at a second location upstream of the test site, the visual label material being delivered to the second strip from the third strip for aqueous wicking delivery to the second strip at the predetermined point in time after the first delivery time.

3. The fluid flow test device of claim 1 wherein the target material is a selected antibody and the capture material is a selected antigen that complexes the selected antibody.

4. The fluid flow test device of claim 2 wherein the second location is upstream of the first location.

5. The fluid flow test device of claim 2 wherein the visual label material is either applied to the third strip of material at the time of use of the test device or the visual label material is pre-applied to third strip of material and embedded therein before use of the test device.

6. Method of testing for the presence or absence of a target material in an aqueous biological sample, comprising:
delivering the aqueous biological sample to a first strip of aqueous wicking material at a first selected delivery time;
providing a second strip of aqueous wicking material having a selected capture material stationarily located at a test site, the capture material being capable of binding to the target material, the first strip of material being in fluid communication with the second strip at a first selected location on the second strip that is upstream of the test site;
delivering a visual label material to the second strip of material upstream of the test site, the visual label material being delivered to the second strip of material at a second predetermined point in time after the first selected delivery time.

7. Method of claim 6 wherein the second predetermined point in time is long enough after the first selected delivery time to ensure that the biological sample has been wicked downstream to the test site.

8. Method according to claim 6 wherein the target material is a selected antibody that binds to the visual label material, the visual label material comprising an antigen that non-selectively binds to the selected antibody and to other antibodies that are contained in the biological sample.

9. Method according to claim 8 wherein the capture material comprises an antigen that selectively binds to the antibody.

10. Method according to claim 6 wherein the visual label material is applied to a third strip of aqueous wicking material that is in fluid communication with the second strip at a second location that is upstream of the first location.

11. Method according to claim 10 wherein a separation distance is selected between the first and second locations such that the biological sample is ensured of being wicked downstream to the test site before the visual label material is wicked downstream to the test site.

12. Method according to claim 6 wherein the visual label material is delivered to a second location on the second strip that is upstream of the first location.

13. Method of testing for the presence or absence of a target material in an aqueous biological sample, comprising:
binding a selected capture material to a test site on a strip of aqueous wicking material at a location downstream of a selected application site on the strip, the capture material being capable of binding to the selected target material;
delivering the aqueous biological sample to the selected application site at a first selected delivery time, and allowing the biological sample to be wicked downstream toward the test site;
delivering a visual label material to the strip of material at a location upstream of the test site for downstream wicking of the visual label material to the test site, the visual label material being delivered to the strip of aqueous wicking material at a second predetermined point in time that occurs after the first selected delivery time.

14. Method according to claim 13 wherein the second predetermined point in time occurs long enough after the first selected delivery time to ensure that the biological sample has been wicked downstream to the test site.

15. Method according to claim 13 wherein the visual label material is delivered to a second location that is upstream of the application site.

16. Method according to claim 15 wherein a separation distance between the application site and the second location is selected such that the biological sample is ensured of being wicked downstream to the test site before the visual label material is wicked downstream to the test site.

17. Method according to claim 13 wherein the target material is a selected antibody that binds to the visual label material, the visual label material comprising an antigen that non-selectively binds to the selected antibody and to other antibodies that are contained in the biological sample.

18. Method according to claim 14 wherein the capture material comprises an antigen that selectively binds to the antibody.

19. A fluid flow test device comprising:
a first strip of aqueous wicking material on which is deposited at a first delivery time an aqueous biological sample to be tested for the presence or absence of a target material;
a second strip of aqueous wicking material having a test site that contains a capture material that binds to the target material, the second strip being in fluid communication with the first strip of aqueous wicking material at a first location upstream of the test site;
a third strip of aqueous wicking material in fluid flow communication with the second strip at a second location upstream of the test site, a visual label material being deposited on the third strip for wicking along the second strip of material to the test site.

20. The fluid flow test device of claim 19 wherein the second location is upstream of the first location a selected distance.

21. The fluid flow test device of claim 20 wherein the distance is selected to ensure that the biological sample is wicked downstream to the test site prior to the visual label material being wicked to the test site when the biological sample is deposited on the first strip at a time that is at least simultaneous with delivery of the visual label material to the second strip.

22. The fluid flow test device of claim 19 wherein the visual label material is delivered to the second strip from the third strip at a predetermined point in time after the first delivery time that is long enough to ensure that the biological sample is wicked downstream to the test site prior to the visual label material being wicked to the test site.

23. The fluid flow test device of claim 19 wherein the target material is a selected antibody and the capture material is a selected antigen that complexes the selected antibody.

24. The fluid flow test device of claim 19 wherein the visual label material is either applied to the third strip of material at the time of use of the test device or the visual label material is pre-applied to third strip of material and embedded therein before use of the test device.

25. A fluid flow test device comprising:
a chromatography path of aqueous wicking material on which is deposited at an upstream site at a selected delivery time an aqueous biological sample to be tested for the presence or absence of a target material;
a capture material bound to the wicking material at a test site downstream of the upstream site, the capture material having a functionality that binds to a first locus on the target material upon wicking of the target material downstream to the test site;
a visual label material having a functionality that binds to a second locus on the target material, the chromatography path having a site upstream of the test site at which the visual label material is begun to wick at a predetermined point in time after the first delivery time such that the visual label material does not intermix with the aqueous biological sample within the chromatography path.

26. The test device of claim 25 wherein the predetermined point in time is selected to be long enough after the selected delivery time to allow the aqueous biological sample to have been already wicked to the test site.

27. The test device of claim 25 wherein the chromatography path comprises a first path extending between the test site and the upstream site at which the sample is applied and a second path at least partially physically separate from the first path extending between the test site and the upstream site at which the visual label material is applied.
